(19) United States
(12) Patent Application Publication
(10) Pub. No.: US 2020/0222538 A1
(43) Pub. Date: Jul. 16, 2020

(54) AUTOMATED TECHNIQUES FOR IDENTIFYING OPTIMAL COMBINATIONS OF DRUGS

(71) Applicant: International Business Machines Corporation, Armonk, NY (US)

(72) Inventors: Marta Sanchez-Martin, Somerville, MA (US); Claudia S. Huettner, Jamaica Plain, MA (US); Jia Xu, Somerville, MA (US); Cheryl L. Eifert, Watertown, MA (US); Elinor Dehan, Haniel (IL); Shang Xue, Cambridge, MA (US); Vanessa Michelini, Boca Raton, FL (US)

(21) Appl. No.: 16/248,068

(22) Filed: Jan. 15, 2019

(51) Int. Cl. A61K 45/06; G16B 5/00; G16B 45/00

(52) U.S. Cl. CPC A61K 45/06; G16B 45/00; G16B 5/00

(57) ABSTRACT

Techniques are provided for administering combination of drug treatments to a patient. Information is analyzed pertaining to individual drug treatments from structurally or functionally defined drugs, drugs with unknown functions, and corresponding effects, wherein the information includes omic data including genes, transcripts, proteins, as well as experimental data from published documents. One or more combinations of drug treatments are identified with combined effects producing a positive result, wherein the positive result is directed to a specific aspect of patient health. The identified combination of drug treatments are administered to a patient.

### 1. TECHNICAL FIELD

Present invention embodiments relate to automated identification of drug combinations, and more specifically, to automated techniques for identifying combinations of drugs to treat biological pathways corresponding to a disease.

### 2. DISCUSSION OF THE RELATED ART

Drug combinations are often superior than single drug treatments for cancer patients. For instance, drug combinations may prevent the development of resistance that may occur during single drug treatments of a disease. However, the biological activity of cancer drugs is usually evaluated as a single agent and predictions of drug combinations that would be advantageous for the patient remain challenging.

In some cases, in vivo experiments may be used to evaluate drug combinations, which are often cumbersome and expensive. If a large number of different drugs are available for combination, the process may be limiting, as it may not be feasible to test all possible combinations in all possible in vivo models.

In other cases, research tools have been developed to predict synergistic interactions between two anticancer drugs. However, these tools typically rely on one source of data, such as expression levels (e.g., protein) or cell treatments with drugs, and usually do not integrate raw data from experiments with data available from publications. Accordingly, it is still difficult to predict whether two drugs will have improved activity in combination.

### SUMMARY

According to embodiments of the present invention, methods, systems and computer readable media are provided for identifying optimal drug combinations. Present techniques may analyze sequencing, transcriptomic and other “omic” data in combination with in vivo literature and clinical data, structure activity drug data (SAR) published or generated by collaborators (e.g., academic institutions or pharmaceutical companies) to identify optimal drug combinations.

Techniques are provided for administering a combination of drug treatments to a patient. Information is analyzed pertaining to individual drug treatments from structurally or functionally defined drugs, drugs with unknown functions, and corresponding effects, wherein the information includes omic data including genes, transcripts, proteins, as well as data from published documents or clinical data. One or more combinations of drug treatments are identified with combined effects producing a positive result, wherein the positive result is directed to a specific aspect of patient health. The identified combination of drug treatments may be administered to a patient by a physician or other health care provider. These techniques allow treatment to be driven by patient-specific omic information to select an optimal combination of drugs.

In one aspect, the one or more combinations of drug treatments is based on biological pathways. This approach allows particular targeting of biological pathways. For example, a first drug of the combination may target a first biological pathway, and a second drug of the combination may target a second biological pathway. In some aspects, the first biological pathway may be in a different biological category than the second biological pathway. By targeting different biological pathways in different biological categories, the therapeutic effect of the drug may be optimized, as the drug combination may target different mechanisms involved in the pathogenesis of the same cancer and may prevent the development of resistance to a single drug.

In other aspects, the first drug and the second drug target a same biological pathway, wherein the first drug is upstream of the second drug. In this case, the patient's cancer is determined to be resistant to the first drug. This approach allows combination therapy when resistance to a drug is suspected or confirmed. The resistance pathway may still be targeted, provided that a drug upstream of the first target is available.

Present techniques determine biological targets from patient-specific data. By obtaining omics information, which may indicate a genetic mutation or other source of the disease, treatment options for that specific mutation or disease may be targeted. In some aspects, the drug combination may be selected based upon corresponding cohort data, wherein the cohort data is similar to the patient-specific data of the patient. For example, for patients having mutations in common, similar diseases, and/or similar medical histories (e.g., age, weight, co-diseases, etc.), a cohort population may be evaluated for optimal drug treatments, instead of the population in aggregate, as specific drugs may have improved performance in subsets of the population instead of the population as a whole.

In a preferred embodiment, the drugs of the drug combination have been approved by a regulatory agency or are in use in clinical trials. Combinations of the drugs may be selected to achieve optimal efficacy while minimizing unwanted side effects.

It is to be understood that the Summary is not intended to identify key or essential features of embodiments of the present disclosure, nor is it intended to be used to limit the scope of the present disclosure. Other features of the present disclosure will become easily comprehensible through the description below.

### BRIEF DESCRIPTION OF THE DRAWINGS

Generally, like reference numerals in the various figures are utilized to designate like components.

FIG. 1 is a block diagram of an example computing environment for drug combination analysis, according to embodiments of the present disclosure.

FIG. 2A is a flowchart for associating drug-based biological targets with biological pathways for drug combination analysis, according to embodiments of the present disclosure.

FIG. 2B is a flowchart for mapping patient-specific data to biological pathway analysis to find drug-based biological targets in biological pathways for drug combination analysis, according to embodiments of the present disclosure.

FIG. 3 is an illustration of different types of omics data that may be provided to the drug combination analyzer, according to embodiments of the present disclosure.

FIG. 4 is an illustration showing an example of different biological pathways and biological categories, according to embodiments of the present disclosure.

FIG. 5 is an illustration showing another example of different biological pathways specific to cancer, according to embodiments of the present disclosure.

FIG. 6 is a flow diagram of determining priority of a drug combination by a scoring module, according to embodiments of the present disclosure.

FIG. 7 is a flow diagram showing operations of the drug combination scoring module, according to embodiments of the present disclosure.

FIG. 8 is a high level flow diagram of operations of the drug combination analyzer, according to embodiments of the present disclosure.

### DETAILED DESCRIPTION

Methods, systems, and computer readable media are provided for identifying optimal combinations of drugs. Present techniques may analyze one or more of sequencing, transcriptomic, proteomic, and other omic data as well as in vitro and in vivo data from experimental treatments with approved or clinical drugs to identify a combination of two or more drugs to treat a medical condition of a patient. Data may be published or generated by any suitable source (e.g., academic institutions, government labs, private or public pharmaceutical companies, etc.).

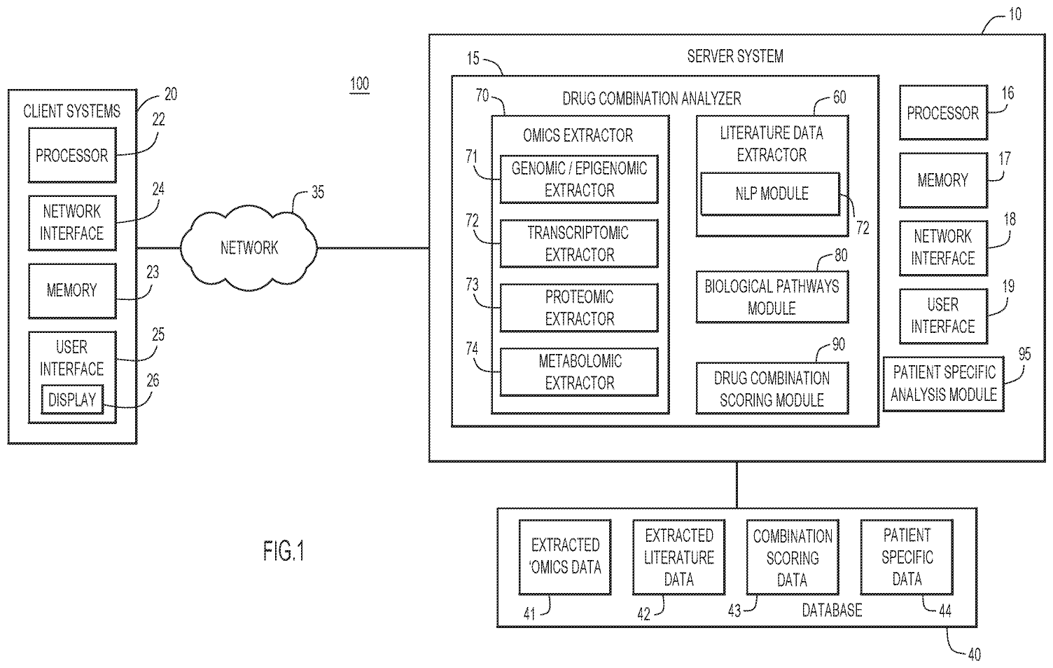

An example environment 100 for use with present invention embodiments is illustrated in FIG. 1. Specifically, the environment includes one or more server systems 10, and one or more client or end-user systems 20. Server systems 10 and client systems 20 may be remote from each other and communicate over a network 35. The network may be implemented by any number of any suitable communications media (e.g., wide area network (WAN), local area network (LAN), Internet, Intranet, etc.). Alternatively, server systems 10 and client systems 20 may be local to each other, and communicate via any appropriate local communication medium (e.g., local area network (LAN), hardwire, wireless link, Intranet, etc.).

Client systems 20 enable users to submit queries (e.g., queries regarding drug combinations for two or more biologic pathways, queries for drug combinations for particular diseases, patient-specific queries to identify optional drug combinations, etc.) to server systems 10 for analysis, to generate a list of drug combinations ranked according to a score. High scoring combinations may be selected for experimental validation and/or therapeutic administration.

A database system 40 may store various information for the analysis (e.g., extracted omics data 41, extracted literature data 42, combination scoring data 43, patient specific data 44, etc.). The database system may be implemented by any conventional or other database or storage unit, may be local to or remote from server systems 10 and client systems 20, and may communicate via any appropriate communication medium (e.g., local area network (LAN), wide area network (WAN), Internet, hardwire, wireless link, Intranet, etc.). The client systems may present a graphical user (e.g., GUI, etc.) or other interface (e.g., command line prompts, menu screens, etc.) to solicit information from users pertaining to the desired query and analysis, and may provide reports including analysis results (e.g., ranked list of drug combinations targeting biological pathways, etc.).

Server systems 10 and client systems 20 may be implemented by any conventional or other computer systems preferably equipped with a display or monitor, a base (e.g., including at least one processor 16, 22, one or more memories 17, 23 and/or internal or external network interfaces or communications devices 18, 24 (e.g., modem, network cards, etc.)), optional input devices (e.g., a keyboard, mouse or other input device), and any commercially available and custom software (e.g., server/communications software, drug combination analyzer 15, browser/interface software, etc.).

Alternatively, one or more client systems 20 may analyze documents to determine drug combination scores when operating as a stand-alone unit. In a stand-alone mode of operation, the client system stores or has access to the data (e.g., extracted omics data 41, extracted literature data 42, combination scoring data 43, patient specific data 44, etc.), and includes a drug combination analyzer 15. The graphical user (e.g., GUI, etc.) or other interface (e.g., command line prompts, menu screens, etc.) solicits information from a corresponding user pertaining to the desired query and analysis, and may provide reports including analysis results (e.g., ranked list of drug combinations targeting biological pathways, etc.).

Extracted omics data 41 and extracted literature data 42 may include extracted information from literature or databases, respectfully, that may indicate the presence of a disease in a patient. For example, this type of data may include genomic mutations associated with diseases, protein expression levels associated with diseases, etc. Combination scoring data 43 may include various drug combinations and their respective scores for treatment of a disease. Patient-specific data 44 may include omic data and other medical history data for a specific patient.

Drug combination analyzer 15 may include one or more modules or units to perform the various functions of present invention embodiments described herein. The various modules (e.g., literature data extractor 60, omics extractor 70, biological pathways module 80, drug combination scoring module 90, etc.) may be implemented by any combination of any quantity of software and/or hardware modules or units, and may reside within memory 17, 23 of the server and/or client systems for execution by processor 16, 22.

Literature data extractor 60 parses literature in machine readable form, e.g., such as scientific publications comprising information including clinical information, etc. to identify information relating to a specific drug for a particular therapeutic target of a biological pathway. In some cases, the literature data extractor 60 may comprise NLP module 72, which may be configured to identify gene names, protein names, drug names, biological targets, efficacies of drugs, drug names, etc., as well as relationships between these entities.

In some aspects, literature data extractor 60 relies on data from in vivo pre-clinical, clinical, and post-clinical studies (instead of just in vitro studies), limiting drugs to those that are approved by a regulatory agency or otherwise available from a clinical trial. Often, the mechanism of the drug is known.

In some cases, the system may be provided with a list of drug names and families of drugs that are approved by the FDA or in clinical trials. For example, the system may be provided with the tradename, generic name, structural name, and/or reference ID (e.g., from a database of drugs) pertaining to the drug, etc. in order to identify and extract relevant information from the literature. In some aspects, the literature data extractor 60 may extract any suitable information to determine an efficacy of a cancer drug including but not limited to statistical values (e.g., mean, median, patient population, p values, etc.), terms pertaining to success of the clinical trial, terms pertaining to failure of the clinical trial, number of clinical trials, phase of clinical trial, etc. In some cases, terms pertaining to the biological target (e.g., protein, cell surface target, cell target, intracellular target, extracellular target, etc.) may also be extracted by the literature data extractor 60, while in other cases, information pertaining to the biological target may be provided by subject matter experts.

Omics extractor 70 may access omic data from various databases (e.g., public, private, etc.), which contain data from genomics, epigenomics, transcriptomics, proteomics, metabolomics, etc. The omics extractor 70 may contain a submodule tailored to extract each type of biological data. For example, genomic/epigenomic extractor 71 may extract and analyze genomic/epigenomic data including genetic alterations and mutations associated with cancer. Transcriptomic extractor 72 may extract and analyze RNA expression profiles, e.g., RNA that are overexpressed, under expressed or remain about the same in cancerous biological samples. Proteomic extractor 73 may extract and analyze protein expression profiles, e.g., proteins that are overexpressed, under expressed or remain about the same, in cancerous biological samples. Similarly, metabolomic extractor 74 may extract and analyze metabolic data. Biological data may include any suitable format, including sequencing data, hybridization microarrays, transcription microarrays, expression microarrays, metabolic microarrays, etc. These modules are described in additional detail through the application.

Biological pathways module 80 maps information from the omics extractor 70 and/or literature data extractor 60 to biological pathways. For example, a first drug may be known to interact with a first entity, and a second drug may be known to interact with a second entity. An entity may be a gene, transcript, protein, metabolite, etc. associated with an omic data set. The biological pathway module 80 may map the first entity to a first biological pathway and the second entity to a second biological pathway. If the first biological pathway and the second biological pathway are not distinct (e.g., the pathways are the same or overlapping), the drug combination analyzer may discard the drug combination. When the first biological pathway is distinct from the second biological pathway, the combination may be passed to the drug combination scoring module 90 for ranking.

Biological pathways module 80 predicts optimal combinations of drugs to target different biological pathways, instead of drugs targeting the same biological pathway. Biological pathways may be determined based on predetermined groups of genes. Drugs may be selected to target different driver genes in different biological pathways.

Drug combination scoring module 90 accepts inputs from the biological pathways module, the literature data extractor, patient-specific analysis module and/or the omics extractor. Based on the received information, the drug combination scoring module ranks the combinations of drugs for a specific patient to provide a list of optimal drug combinations, which may be stored as combination scoring data 43.

Patient-specific analysis module 95 receives input data from client system 20 which may include omics data of a specific patient. This information may be parsed and provided to the biological pathways module, allowing combinations of drugs targeting different relevant biological pathways to be identified based upon the patient-specific data, which may be stored as patient-specific data 44. This data may be used to tailor the drug combination to a specific patient based on omics and other data (e.g., tumor type, tumor mutation, clinical data, medical history, etc.).

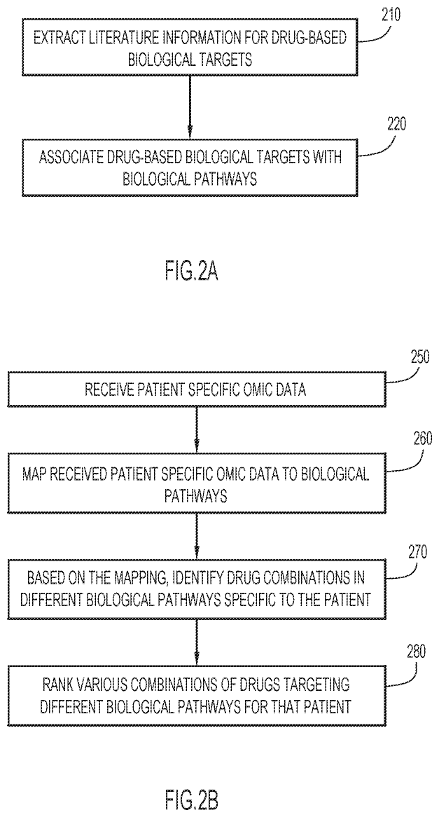

FIG. 2A is a flow diagram showing association of cancer targets with biological pathways. At operation 210, literature data is extracted from databases, scientific literature, and clinical and preclinical literature, as well as any other source of relevant information, which relates to biological targets of specific drugs that are in clinical trials or that have been approved by regulatory agencies. In some cases, this information includes a drug interaction with a specific biological molecule of a pathway (e.g., evidence that a drug binds to a biological molecule, inhibits a biological pathway, activates a biological pathway, etc.). At operation 220, the biological targets are associated with biological pathways. This sets up a framework to provide optimal drug combinations. For example, the system may map specific biological targets of drugs to a specific biological molecule of a biological pathways or to the biological pathway in general to select drugs for a patient that target specific pathways. In some aspects, the biological effects of an approved or experimental (e.g., in clinical or pre-clinical trials) anti-tumor drug may be extracted or derived from clinical trials, or other experimental results (e.g., animal models, in silico data, etc.).

FIG. 2B shows a flow diagram of ranking various combinations of drugs based on patient specific data. At operation 250, patient specific omic data is obtained. This may include protein expressions levels (e.g., including one or more biomarkers associated with cancer), genomic sequences (e.g., including mutations that are associated with cancer, presence of specific driver genes that are associated with cancer, etc.), RNA expression levels (e.g., including specific transcripts associated with cancer), etc. In some aspects, the omic data may be analyzed by the omic service provider (e.g., a company performing genomic sequencing and/or offering microarray analysis or other services to evaluate gene translation, protein expression, etc.) A report may be provided to the patient or medical provider regarding the results of the report, and may identify a genomic mutation, altered protein expression levels as compared to a control without cancer, altered transcription profiles as compared to a control without cancer, etc.). At operation 260, the system may map the received patient specific omic to biological pathways. For example, if the patient specific data shows a mutation in a particular protein of a biological pathway, the system will identify the protein's presence in a biological pathway. If the omics data indicated an effect of a biological pathway, the pathway may be identified as a target (instead of a particular molecule of a pathway). At operation 270, the system may identify drug combinations in different biological pathways suitable for treatment of the patient. For example, if the omics mutation indicates that a cell growth mutation has occurred that promotes proliferation, the system may determine whether drug combinations having at least one drug in the cell growth category are present. The various combinations may be provided to scoring module 90, at operation 280, for ranking of the specific combinations.

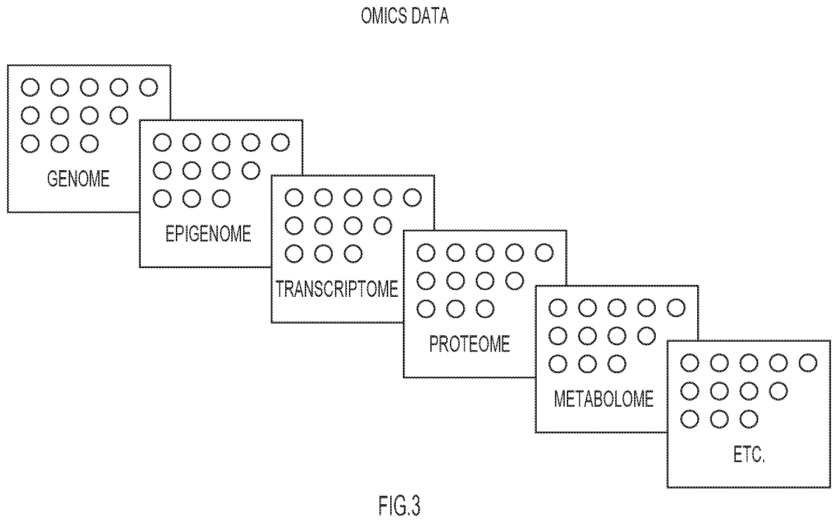

FIG. 3 shows omics data that may include but is not limited to data from genomics, epigenomics, transcriptomics, proteomics, metabolomics, etc. studies. In some aspects, omics data may be obtained from publicly available databases, which may include publications, sequences, expression or transcription levels from microarray analyses, other results of omic studies, etc.

Accordingly, for each of these categories, the data may be analyzed to identify various cancer related targets. For example, genomic/epigenomic data may be analyzed to identify genes and mutations associated with cancer, as well as transcription and expression levels of molecules involved in the development and pathogenesis of cancer. Certain types of cancer may have specific transcription or expression profiles, which are associated with a biological pathway.

Thus, omics extractor 70 may identify specific information (e.g., mutations, transcription profiles, expression profiles, etc.) that are associated with specific types of cancer. This information may be stored as extracted omics data 41. When patient specific information is provided to the system, the system may utilize the extracted omics data to identify patient specific data associated with particular types of cancer. Based on this information, specific biological targets and/or pathways may be identified as potential drug targets.

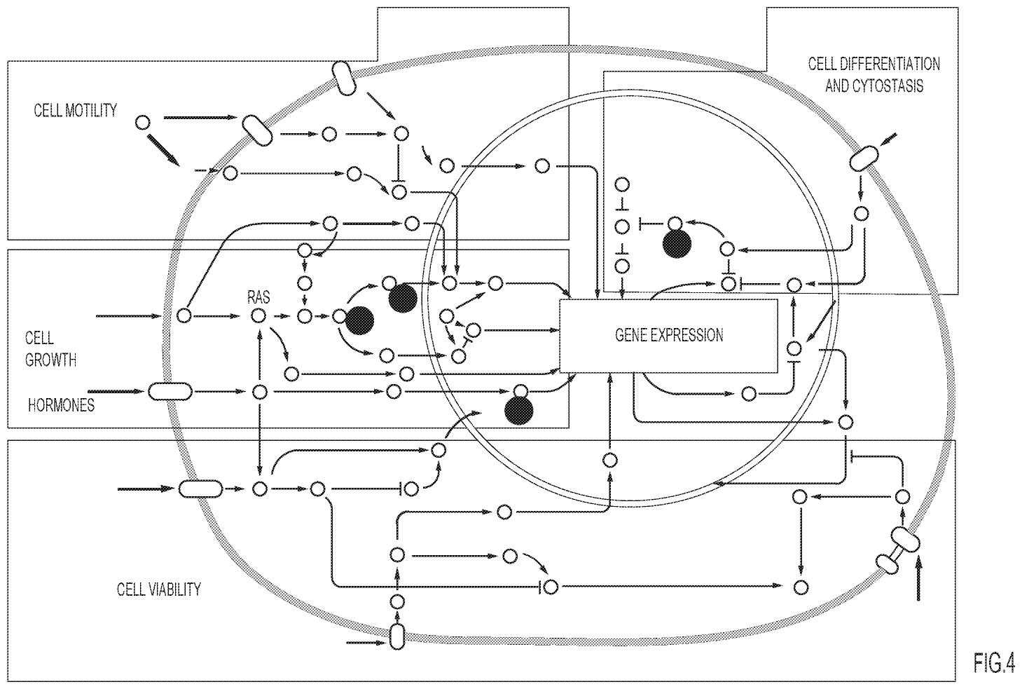

FIG. 4 shows various biological pathways. In this example, the pathways are shown arranged according to categories including cell motility, cell growth, cell viability, and cell differentiation and cytostasis. The nodes represent various entities (e.g., proteins, chemical molecules, etc.) in the pathway which have a particular biological structure. The black arrows show interconnectivity between nodes of biological pathways. In this example, the outer circle represents an outline of the cell, whereas the inner circle represents an outline of the nucleus.

A variety of biological pathways may be targeted in a variety of manners including extracellularly, at the cell membrane, inside the cell at the cytoplasmic level, as well as inside the nucleus which controls gene expression.

For the purposes of this example, solid circles are shown which correspond to various potential biological targets of drugs. For example, in the cell growth category, two solid circles are present along a same biological pathway (e.g., a Ras pathway). In this case, the drug analyzer would consider this combination of drugs to be redundant (and such combinations may optionally be excluded), unless resistance was indicated in the upstream target, then the downstream target may still be selected. On the other hand, a drug targeting cell differentiation and cell growth would cover different biological pathways, and the system may provide this combination to the ranking module for further analysis. Additionally, targets in the same category may be considered distinct, e.g., the cell growth category along a hormone-based pathway and the Ras pathway of the cell growth category may be considered distinct and provided to the ranking module for further analysis.

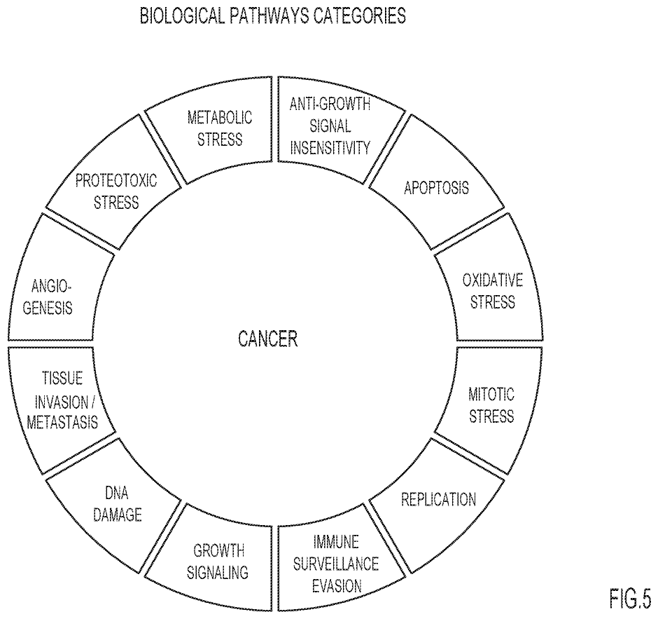

FIG. 5 shows a general overview of various biological pathway categories associated with cancer. These include immune surveillance evasion, angiogenesis, apoptosis, growth signaling, cell replication, metastasis and tissue invasion, DNA damage, mitotic stress, proteotoxic stress, metabolic stress, oxidative stress, anti-growth signal insensitivity, differentiation, etc. Any of these categories may be targeted, may be used to select a drug targeting a first pathway in combination with another drug targeting a second pathway, to find combinations having an improved efficacy than either drug alone.

The examples of FIG. 4 or FIG. 5 are not intended to be limited to the categories or biological pathways represented in these examples. Any suitable pathway may be shown.

In some aspects, the combination of drugs targeting different biological pathways may be synergistic, such that the effect of the combined drugs is greater than each drug administered separately and combined additively. In other cases, the combination of drugs targeting different biological pathways may be additive, such that the effect of the combined drugs is the same or similar to the sum of the effects of each drug administered separately.

The data may be combined for each drug combination of two or more drugs, generating a score for each drug combination. The highest scoring combinations may be identified as candidates for administration to a patient.

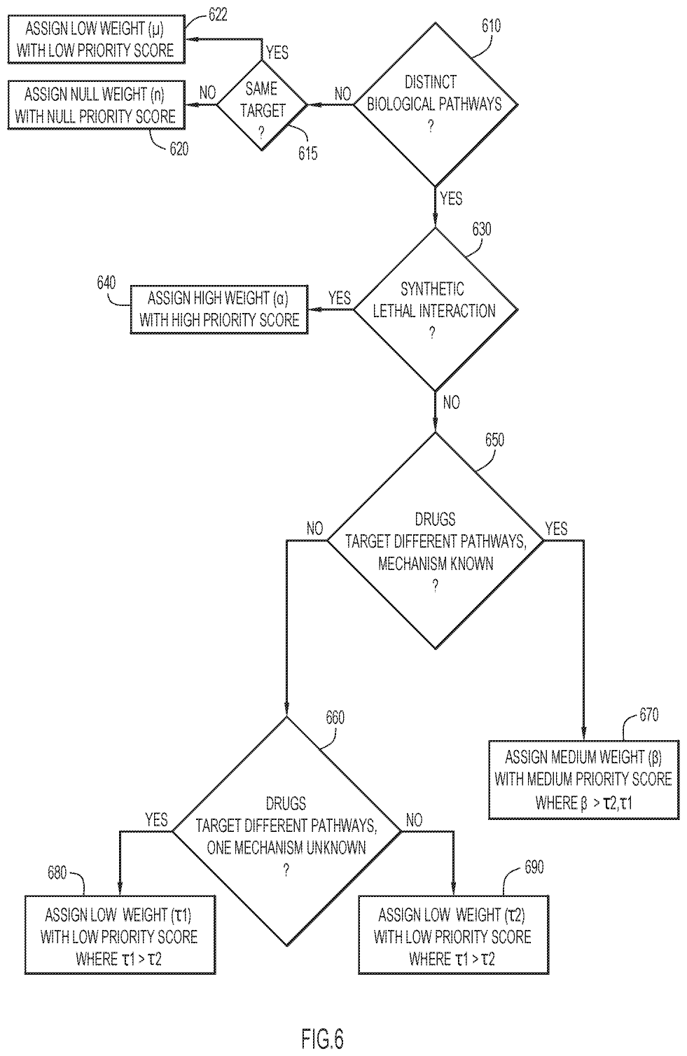

FIG. 6 shows a ranking module that provides a patient-centric ranking system of FDA-approved or investigational drugs in clinical development. The ranking module is predictive in the regard that it predicts the best combinations of drugs, wherein the drugs are approved by a regulatory agency or are in clinical trials. In some aspects, the ranking is based on patient-specific data that includes omic information, clinical data (e.g., stage of disease, type of cancer, etc.), and other health-related data about the patient as well as clinical data of various drug efficacies that have been approved or are in clinical trials. In some cases, a patient's omic data may be used to identify specific biological pathway targets in order to identify optimal drug combinations.

Drug combination scoring module 90 ranks the results based upon the strength of evidence-based data. Features used to rank the results may include the patient's omic data (e.g., genomic profile, etc.), patient clinical data (e.g., stage of disease, type of cancer, etc.), drug characteristics (e.g., specificity of the drug relative to the patient's mutation(s), efficacy of the drug, drugs described to have a synergistic relationship in the literature, etc.), sample size of the evidence-based data, etc. Ranking may rely on the reported outcome of patients with common biomarkers (mutations). The mechanism of action is often known and used for ranking of treatment options.

Drug combinations and ranking of drugs is based on published clinical data in patients associated with a particular mutation and possibly in combination with another drug targeting a different mutation, affecting two or more oncogenic pathways. Drug combinations are expected to be well-tolerated, and in many cases safe doses and toxicities of the individual drugs are known from the literature or clinical trials.

FIG. 6 shows an example method of assigning a priority to a drug combination. In one aspect, ranking may be governed in part by determining a priority score of the drug combination. At operation 610, the scoring module evaluates whether the biological pathways are distinct. If the pathways are not distinct, the system may determine whether the drugs are directed towards the same target. At operation 615, if the drugs are directed toward the same target, a null priority score may be assigned to the combination at operation 620. In this case, the weighting factor (n) is zero, as the targets are the same. In other cases, the two drugs may be directed towards different targets within the same biological pathway. For instance, a first target may be upstream of a second target along the same biological pathway, and the first target may have developed resistance to the therapy. In this case, at operation 622, a low weight (μ) may be assigned, which leads to a low priority score of the drug combination. In some cases, the low weight (μ) may be less than other weights (e.g., τ2, τ1).

At operation 610, if the biological pathways are distinct, then the system progresses to operation 630. At operation 630, the system assesses whether the combination of drugs has a synthetic lethal interaction. If two drugs targeting two different pathways are identified in the literature as having a synthetic lethal interaction (e.g., blocking both biological targets leads to cell death), then the system progresses to operation 640 and a high weight (a) is assigned to generate a high priority score. If one target is blocked, this effect is much less strong than a lethal defect.

In the absence of a synthetic lethal interaction, the system progresses to operation 650, wherein the drugs target different pathways having mutations designated as relevant for the disease. If the mechanism of both drugs are known, then the system progresses to operation 670. A medium weight (β) is assigned to generate a medium priority score, where β>τ2, τ1. For example, two drugs (e.g., a biologic, a small molecule, etc.) may be determined to work well together.

If the mechanism of one or both drugs are unknown, then the system progresses to operation 660. If only one mechanism is unknown, the system progresses to operation 680, wherein a low weight (τ1) is assigned to generate a low priority score, where τ1>τ2. Otherwise, at operation 690, two drugs with unclear mechanisms of action (unclear biological pathways) may be presumed to target redundant pathways, and assigned a low priority score (τ2), where τ1>τ2.

Thus, this method provides an example of how strength of evidence-based data (e.g., specificity of the drug relative to the patient's mutation(s), efficacy of the drug, sample size of the evidence-based data, etc.) may be used to rank drug combinations. A patient-centric ranking may be based on clinical data and a correlation with the patient's disease.

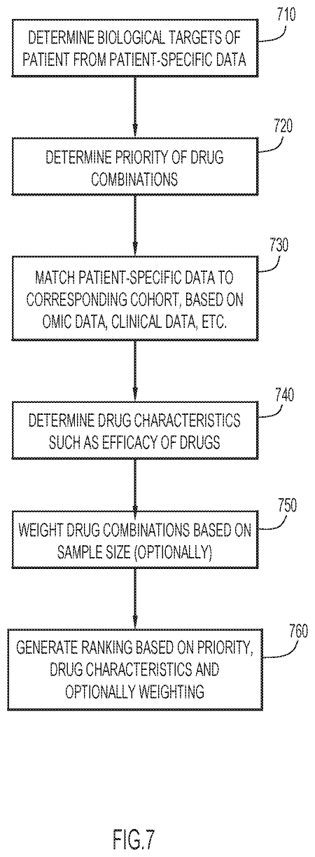

FIG. 7 is a flow chart showing aspects of ranking drug combinations. At operation 710, biological targets of the patient are determined from patient-specific data. At operation 720, the priority of drug combinations are determined. At operation 730, patient-specific data is matched to a corresponding cohort, based on omic data, clinical data, etc. At operation 740, drug characteristics such as efficacy of drugs are determined (e.g., based on ED50, LD50 values), optionally with respect to a cohort. At operation 750, drug combinations may be weighted based on sample size at 750. Any suitable statistical weighting technique may be used including frequency weights, survey weights, analytical weights, importance weights, sample size weights, etc. For example, at operation 760, ranking is generated based on priority, drug characteristics, and optionally sample size weighting. For example, priority may be weighted based on predicted combination strength. For two drugs, various combinations may be evaluated: two drugs targeting synthetic lethal interactions >two drugs targeting two pathways and described as synergistic in the literature >two drugs targeting the same pathway. Points or weightings may be assigned based on this ranking such that two drugs having synthetic lethal interactions (top priority) may be assigned a priority of +4 points, two drugs predicted to have a synergistic interaction (intermediate priority) may be assigned a priority of +2 points, and two drugs targeting the same pathway with no predicted synergy (low priority) may be assigned a priority of +1 point. Drug characteristics may also be assigned points based whether or not each drug of the drug combination is approved by a regulatory agency. For example, for a case with two drugs, various combinations may be evaluated: both drugs approved by a regulatory agency >one drug approved by a regulatory agency >both drugs are investigational. Points or weightings may be assigned based on this ranking such that both drugs approved (top) may be assigned a drug characteristic of +3 points, both drugs approved (intermediate) may be assigned a drug characteristic of +2 points, and neither drug approved (low) may be assigned a drug characteristic of +1 point. A similar point assignment may be used for sample size, e.g., assigning points to various sample size ranges to favor larger sample sizes over smaller sample sizes. Any point range may be used, e.g., any negative, neutral (zero), or positive number may be used, and the point range is not intended to be limited based on this example.

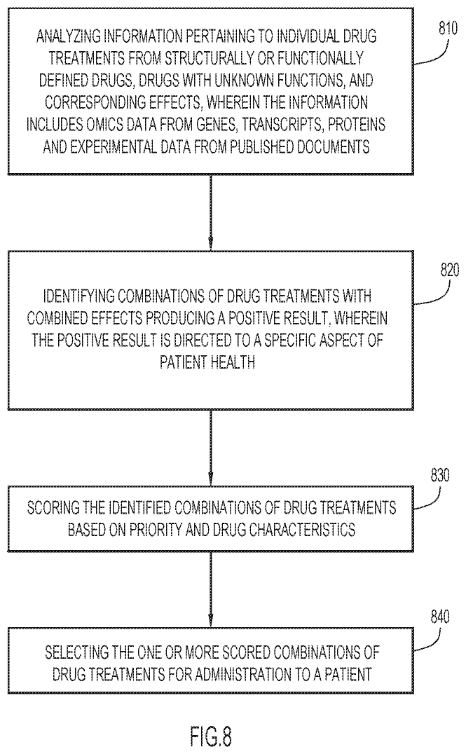

FIG. 8 is a flow chart showing high level operations of the present system. At operation 810, information is analyzed pertaining to individual drug treatments from structurally or functionally defined drugs, drugs with unknown functions, and corresponding effects, wherein the information includes genes, transcripts, and published documents. Present techniques use clinical and preclinical data from the published literature to identify optimal treatment options for cancer patients based on the omic profile of the tumor. At operation 820, one or more combinations of drug treatments are identified with combined effects producing a positive result, wherein the positive result is directed to a specific aspect of patient health. In many cases, the mechanism of action of the drug is known and used for ranking of treatment options. Ranking may be based on the evidence based data (see, FIG. 6, etc.) as well as other factors including reported outcome of patients (e.g., a similar cohort) having the same biomarkers (mutations). The present techniques identify the most promising candidates among a large array of compounds. At operation 830, an identified combination of drug treatments is administered to a patient.

Present techniques may be used to select combinations of compounds for preclinical and clinical development, thereby reducing costs and saving time on expensive in vivo or in vitro experiments. These techniques may identify the most promising candidates among a large array of approved or investigational compounds, and may identify new drug combinations for a broad range of diseases.

Present techniques may be used to identify new combinations of approved or clinical drugs, e.g., based on targeting of biological pathways. In some cases, the new combinations may arise from selecting targets on different biological pathways. In other cases, the new combinations may arise from selecting targets to combat resistance (e.g., selecting a downstream target of a biological pathway when the upstream target is suspected or confirmed to be resistant to a treatment.

In some embodiments, sensors may be embedded in a patient, wherein the sensor comprises a cancer monitor/sensor that measures cancer biomarkers or other biological analytes that are indicative of the presence, and preferably, amount of cancer. For example, if a drug combination is administered, the cancer sensor may detect a decrease in the biological analyte, which would indicate that the administered therapy is effective in treatment of the cancer. However, if the cancer acquires resistance, then the cancer may grow and the biological analyte level may increase. In this case, the sensor may alert the physician that a change in therapy should be considered, as the cancer is becoming or has become resistant to the administered therapy.

Other advantages of present techniques include using an evidence-based approach that relies on data from pre-clinical, clinical, and post-clinical studies (instead of in vitro studies). Evaluating combinations of drugs to target different biological pathways, instead of drugs targeting the same biological pathway, is predicted to produce an optimal therapeutic effect. Biological pathways may be predetermined based on groups of genes (e.g., with reference to FIG. 4, a biological pathway may be a series of connected nodes corresponding to a series of proteins produced from expression of respective genes) that produce a biological effect), and drugs may be selected to target particular genes (e.g., driver genes, mutated proteins, overexpressed proteins, etc.) in different biological pathways. In some cases, drugs are limited to those with a known mechanism and that are approved by a regulatory agency or otherwise available from a clinical trial. Tailoring the treatment to a specific patient based on patient-specific data (e.g., tumor type, tumor mutation, clinical or other medical information, etc.) allows for an optimal combination to be predicted.

The environment of the present invention embodiments may include any number of computer or other processing systems (e.g., client or end-user systems, server systems, etc.) and databases or other repositories arranged in any desired fashion, where the present invention embodiments may be applied to any desired type of computing environment (e.g., cloud computing, client-server, network computing, mainframe, stand-alone systems, etc.). The computer or other processing systems employed by the present invention embodiments may be implemented by any number of any personal or other type of computer or processing system (e.g., desktop, laptop, PDA, mobile devices, etc.), and may include any commercially available operating system and any combination of commercially available and custom software (e.g., browser software, communications software, server software, drug combination analyzer 15, etc.). These systems may include any types of monitors and input devices (e.g., keyboard, mouse, voice recognition, etc.) to enter and/or view information.

It is to be understood that the software (e.g., drug combination analyzer 15, including omics extractor 70, literature data extractor 60, biological pathways module 80, and drug combination scoring module 90, etc.) of the present invention embodiments may be implemented in any desired computer language and could be developed by one of ordinary skill in the computer arts based on the functional descriptions contained in the specification and flow charts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client and server systems, and/or any other intermediary processing devices. The software and/or algorithms described above and illustrated in the flow charts may be modified in any manner that accomplishes the functions described herein. In addition, the functions in the flow charts or description may be performed in any order that accomplishes a desired operation.

The software of the present invention embodiments (e.g., drug combination analyzer 15, including omics extractor 70, literature data extractor 60, biological pathways module 80, and drug combination scoring module 90, etc.) may be available on a non-transitory computer useable medium (e.g., magnetic or optical mediums, magneto-optic mediums, floppy diskettes, CD-ROM, DVD, memory devices, etc.) of a stationary or portable program product apparatus or device for use with stand-alone systems or systems connected by a network or other communications medium.

The communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information (e.g., extracted omics data 41, extracted literature data 42, combination scoring data 43, patient specific data 44, reports indicating optimal drug combinations based on patient specific data, etc.). The database system may be implemented by any number of any conventional or other databases, data stores or storage structures (e.g., files, databases, data structures, data or other repositories, etc.) to store information (e.g., extracted omics data 41, extracted literature data 42, combinations scoring data 43, patient specific data 44, reports indicating optimal drug combination based on patient specific data, etc.). The database system may be included within or coupled to the server and/or client systems. The database systems and/or storage structures may be remote from or local to the computer or other processing systems, and may store any desired data (e.g., extracted omics data 41, extracted literature data 42, combinations scoring data 43, patient specific data 44, reports indicating optimal drug combination based on patient specific data, etc.).

The present invention embodiments may employ any number of any type of user interface (e.g., Graphical User Interface (GUI), command-line, prompt, etc.) for obtaining or providing information (e.g., extracted omics data 41, extracted literature data 42, combinations scoring data 43, patient specific data 44, reports indicating optimal drug combination based on patient specific data, etc.), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

The report may include any information arranged in any fashion, and may be configurable based on rules or other criteria to provide desired information to a user (e.g., text analytics, drug combination scores, patient-specific information, etc.).

The present invention embodiments are not limited to the specific tasks or algorithms described above, but may be utilized for any medical condition in which combination therapy is desired, and information pertaining to efficacy of individual therapies are available. These techniques may be applied to any quantity of drugs in a combination.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method of selecting a combination of drug treatments for a given patient comprising:
analyzing, using a drug combination analyzer system comprising at least one processor and at least one memory, information pertaining to individual drug treatments from structurally or functionally defined drugs, drugs with unknown functions, and corresponding effects, wherein the information includes genes, transcripts, and published documents;
identifying, using the drug combination analyzer system, one or more combinations of drug treatments with combined effects producing a positive result, wherein the positive result is directed to a specific aspect of patient health;
scoring the identified combinations of drug treatments based on priority and drug characteristics; and
selecting, using the drug combination analyzer system, the one or more scored combinations of drug treatments for administration to a patient based on biological pathways.

2. The method of claim 1, wherein a first drug of a combination targets a first biological pathway, and a second drug of the combination targets a second biological pathway.

3. The method of claim 2, wherein the first biological pathway is in a different biological category than the second biological pathway.

4. The method of claim 1, wherein the first drug and the second drug target a same biological pathway, and wherein the first drug is upstream of the second drug.

5. The method of claim 4, wherein a patient's cancer is determined to be resistant to the first drug.

6. The method of claim 1, wherein the combinations are selected to target specific biological molecules determined from patient-specific data.

7. The method of claim 1, further comprising selecting the combinations based upon corresponding cohort data, wherein the cohort data is similar to the patient-specific data of the patient.

8. A system of selecting a combination of drug treatments for a given patient comprising at least one processor configured to:
analyze, using a drug combination analyzer system comprising at least one processor and at least one memory, information pertaining to individual drug treatments from structurally or functionally defined drugs, drugs with unknown functions, and corresponding effects, wherein the information includes genes, transcripts, and published documents;
identify, using the drug combination analyzer system, one or more combinations of drug treatments with combined effects producing a positive result, wherein the positive result is directed to a specific aspect of patient health;
score the identified combinations of drug treatments based on priority and drug characteristics; and
select, using the drug combination analyzer system, the one or more scored combinations of drug treatments for administration to a patient based on biological pathways.

9. The system of claim 8, wherein a first drug of a combination targets a first biological pathway, and a second drug of the combination targets a second biological pathway.

10. The system of claim 9, wherein the first biological pathway is in a different biological category than the second biological pathway.

11. The system of claim 8, wherein the first drug and the second drug target a same biological pathway, and wherein the first drug is upstream of the second drug.

12. The system of claim 11, wherein a patient's cancer is determined to be resistant to the first drug.

13. The system of claim 8, wherein the combinations are selected to target specific biological molecules determined from patient-specific data.

14. The system of claim 8, further comprising selecting the combinations based upon corresponding cohort data, wherein the cohort data is similar to the patient-specific data of the patient.

15. A computer program product for selecting a combination of drug treatments for a given patient, the computer program product comprising a computer readable storage medium having program instructions embodied therewith, the program instructions executable by a computer to cause the computer to:
analyze, using a drug combination analyzer system comprising at least one processor and at least one memory, information pertaining to individual drug treatments from structurally or functionally defined drugs, drugs with unknown functions, and corresponding effects, wherein the information includes genes, transcripts, and published documents;
identify, using the drug combination analyzer system, one or more combinations of drug treatments with combined effects producing a positive result, wherein the positive result is directed to a specific aspect of patient health;
score the identified combinations of drug treatments based on priority and drug characteristics; and
select, using the drug combination analyzer system, the one or more scored combinations of drug treatments for administration to a patient based on biological pathways.

16. The computer program product of claim 15, wherein a first drug of a combination targets a first biological pathway, and a second drug of the combination targets a second biological pathway.

17. The computer program product of claim 16, wherein the first biological pathway is in a different biological category than the second biological pathway.

18. The computer program product of claim 15, wherein the first drug and the second drug target a same biological pathway, and wherein the first drug is upstream of the second drug.

19. The computer program product of claim 18, wherein a patient's cancer is determined to be resistant to the first drug.

20. The computer program product of claim 15, wherein the combinations are selected to target specific biological molecules determined from patient-specific data.
